(19) United States
(12) Patent Application Publication
(10) Pub. No.: US 2023/0288417 A1
(43) Pub. Date: Sep. 14, 2023

(54) METHOD AND KIT FOR VIRUS IDENTIFICATION

(71) Applicant: Idea Bio-Medical Ltd., Rehovot (IL)

(72) Inventors: Yael PARAN, Moshav Beit Oved (IL); Shlomo TURGEMAN, Rishon Letzion (IL); Daniel Feigelson, Rehovot (IL)

(21) Appl. No.: 17/801,511

(22) PCT Filed: Feb. 22, 2021
(86) PCT No.: PCT/IB2021/051492

§ 371 Date: Aug. 22, 2022

Related U.S. Application Data
(60) Provisional application No. 63/049,629, filed on Jul. 9, 2020.
(60) Provisional application No. 62/987,346, filed on Mar. 9, 2020.
(60) Provisional application No. 62/981,077, filed on Feb. 25, 2020.
(60) Provisional application No. 62/979,763, filed on Feb. 21, 2020.

(30) Foreign Application Priority Data
Aug. 18, 2020 (IL) 276771

(51) Int. Cl. G01N 33/569

(52) U.S. Cl. CPC G01N 33/56983; G01N 2333/165; G01N 2470/04

(57) ABSTRACT

There is provided a method for detecting the presence of cells infected with a coronavirus. A sample obtained from a patient is transferred to a sample vessel and labeled molecules that binding different viral structural proteins are incubated with the sample. An apparatus useful for practicing the method is also discussed. Other embodiments are also disclosed.

This application claims priority from, and as appropriate the benefit of, U.S. Provisional Applications Nos. 62979763, 62981077, 62987346, and 63049629, filed February 21, February 25, March 9 and Jul. 9, 2020, respectively, and Israel Patent Application no. 276771 filed Aug. 18, 2020. These applications are incorporated herein by reference in their entireties.

Presently, to test whether or not a patient is infected with a virus, it is necessary to obtain a sample from a patient and then manipulate the sample in a laboratory. Such manipulation may require (1) growing cells, either growing the original sample cells or growing other cells which are contacted with the original cells so as to become infected with the virus if some of the sample cells are themselves infected, (2) subsequently testing for the presence of the virus, either (a) directly using a quantity of an antibody that binds to the virus itself or binds to a cell-surface antigen indicative of infection of the cell by the virus (e.g. a major histocompatibility complex protein presenting an epitope of the viral protein), and then confirming, often spectrophotometrically, whether or not the antibody has bound the targeted viral or epitope-presenting proteins; or by (b) using the polymerase chain reaction (PCR) to increase the amount of viral DNA or RNA present and then testing for the presence of one or more DNA or RNA sequences known to be unique to the virus.

Regardless of how viral testing is conducted, the process is time-consuming, normally requiring at least several hours, and having limited throughput because of the need for the aforementioned laboratory manipulation. If the patient is not located near a laboratory, then additional time is required, either to transport the patient to a laboratory facility, or, if the patient is sampled at a location remote from a laboratory, to transport the sample to a suitable laboratory.

There is provided in accordance with one embodiment of the present invention a method for testing a mammalian patient for the presence of a virus in the patient. The method involves 2. transferring to a sample vessel a sample which has been obtained from a patient, which sample after being obtained from the patient and prior to being transferred to the sample vessel has not been (a) incubated with other cells (b) diluted, including diluted by contacted with a fixing solution; (c) frozen or (d) centrifuged, the sample vessel being substantially free of cells other than those in the sample;
2A. fixing the cells of the sample by contacting them with a fixing solution for a time sufficient to fix the cells in the sample vessel, and thereafter conducting an initial washing the fixed cells with a washing solution;
2B. optionally, contacting the sample with a blocking protein, and thereafter washing the sample;
2C. optionally, contacting the sample in the vessel with a permeabilization agent, and thereafter washing the sample;
wherein said contacting the sample with a permeabilization agent, if conducted, is conducted simultaneously with or subsequent to said contacting with a fixing solution, and wherein said contacting with a blocking protein, if conducted, is conducted simultaneously with or subsequent to said contacting with a fixing solution; and wherein, if said contacting the sample with a permeabilization agent is conducted simultaneously with said contacting with a fixing solution, said conducting an initial washing and said thereafter washing are done as a single washing step; and wherein, if said contacting the sample with a blocking protein is conducted simultaneously with said contacting with a fixing solution, said conducting an initial washing and said thereafter washing are done as a single washing step;
3. contacting the sample in the sample vessel with a first antibody known to bind a viral substrate, the viral substrate being at least one of (i) an intact first viral protein and (ii) a portion of a first viral protein when presented as part of a cell-surface antigen,
4. after an incubation period to allow binding of the first antibody to the viral substrate, conducting a first washing of the sample in the sample vessel
5. if the first antibody is not a labeled with a first label, after the first washing contacting the first antibody with a second antibody that binds the first antibody, the second antibody being labeled with a first label, and after an incubation period to allow binding of the second antibody to the first antibody, washing the sample in the sample vessel;
6. inducing conditions which will cause the first label, if present, to produce a first signal
7. during the time period in which the first signal would be produced if the first label is present in the sample, capturing at least one first image of the sample in the sample vessel,
8. at least one of (a) sending the at least one first image to a remote analysis station, and (b) analyzing the at least one first image to determine the presence or absence of the first signal, wherein the presence of the first signal indicates presence of the virus in the sample.

In some embodiment, prior to number 8 above, the method includes the following steps:
3A. contacting the sample in the sample vessel with a third antibody known to bind a viral substrate, the viral substrate being at least one of (i) an intact second viral protein and (ii) a portion of a second viral protein when presented as part of a cell-surface antigen, wherein said second viral protein is different from said first viral protein;
4A. after an incubation period to allow binding of the third antibody to the viral substrate, conducting a second washing of the sample in the sample vessel
5A. if the third antibody is not labeled with a second label, after the second washing contacting the third antibody with a fourth antibody that binds the second antibody, the fourth antibody being labeled with a second label, and after an incubation period to allow binding of the fourth antibody to the third antibody, washing the sample in the sample vessel;
6A. inducing conditions which will cause the second label, if present, to produce a second signal
7A. during the time period in which the second signal would be produced if the second label is present in the sample, capturing at least one second image of the sample in the sample vessel,
8A. at least one of (a) sending the at least one second image to a remote analysis station, and (b) analyzing the at least one second image to determine the presence or absence of the second signal, wherein the presence of the second signal indicates presence of the virus in the sample.

In some embodiments, 3 and 3A are carried out simultaneously; 4 and 4A are carried out simultaneously, viz. the first and second washing are a single washing; 5 and 5A, if carried out, are carried out simultaneously, and excess third and fourth antibodies are washed out in a single washing step; 7 and 7A are carried out simultaneously, and the at least one first image and the at least one second image are the same at least one image; and 8 and 8A are carried out simultaneously. In such embodiments, the presence of both the first and second signals in the image is a better indication of the presence of the virus than the presence of only the first signal or only the second signal.

In a variation on the embodiments described above, the method involves the steps described above, but is varied in that (i) between being obtained and prior to transferring to the sample vessel, the sample is contacted with a fixing solution, but is not (a) incubated with other cells (b) diluted with a solution other than a fixing solution; (c) frozen or (d) centrifuged, and the sample vessel is substantially free of cells other than those in the sample, (ii) step 3A is omitted; and (iii) the method utilizes both the first antibody and the third antibody.

In some embodiments, the virus is a coronavirus. In some embodiments, the coronavirus is the SARS Cov2 virus or a variant thereof.

In some embodiments, the method further comprises obtaining the sample. In some embodiments, when the patient is human, it is expected that the patient may obtain its own sample, for example by swabbing the patient’s mouth, throat, or nasal passage using a cotton-tipped swab, although the sampling may also be conducted by a third party, e.g. a physician, or a veterinarian in the case of a non-human patient, and the sample may be collected in other ways or from other parts of the body, for example by collection of a fluid such as saliva, blood, or mucus, or by obtaining a stool sample. Additionally, it is expected that in some cases, the patient will transfer the sample to the sample vessel, although this too may be done by a third party.

The sample vessel should be such that the sample, or a portion thereof, may easily be transferred to the vessel, and should present an open area that facilitates easy image acquisition. Examples of such sample vessels are 96- or 384-well plates that are commonly used in laboratories, a Petri dish, or a microscope slide. It may be desirable to treat the surface of the vessel prior to introduction of the sample so as to increase the adhesion of the cells to the vessel surface. For example, if the vessel is made of glass, it may be helpful to first coat the vessel with polylysine. In cases in which a sample has been obtained by swabbing, transfer of the sample to the sample vessel may be effected, for example, by wiping the sample-containing swab on the sample vessel surface, optionally with rolling of the swab so as to increase the degree of transfer from the swab to the vessel. In cases in which the sample is a liquid sample, e.g. saliva or mucus, a small amount of the sample may be applied to the surface of the sample vessel, for example by pouring, by ejecting from a syringe, or by using a cotton-tipped swab, optionally followed by spreading of the sample on the sample vessel surface.

Following transfer of the sample to the sample vessel, the cells therein are “fixed”, viz they are contacted with a fixation agent that terminates any ongoing biochemical reactions in the cells in the sample and preferably increases the mechanical strength or stability of the cells. An example of a common fixing agent is paraformaldehyde and Tween®20 in phosphate buffer. As noted above, if both the first and third antibodies are incubated with the sample, then the sample may be fixed prior to transfer to the sample vessel.

Once the sample or portion thereof has been transferred to the sample vessel and the cells fixed, and fixation solution, if present (and/or blocking protein and/or permeabilization agent, if present, as discussed below), washed away, cells from the patient present in the sample are tested for the presence of the virus. While in principle there are a number of ways this may be accomplished, embodiments of the present invention contemplate, as a first step in doing so, contacting the sample in or on the sample vessel with an antibody that binds to the viral substrate, viz. to an intact viral protein, or to a portion of such a viral protein when presented as part of a cell-surface antigen, e.g. presented by an MHC-1 protein. As used herein, when it is stated that an antibody binds to an intact viral protein, it to be understood that the antibody does not necessarily bind to the entirety of the viral protein, but rather to at least a portion of the viral protein that is exposed to the antibody, that portion being in its native three-dimensional conformation as part of an intact viral protein. Typically this contacting is accomplished by introducing a small amount of a solution of containing the antibody to the sample vessel. The antibody that binds the viral substrate may be labelled, so that the bound antibody’s presence may be detected. Examples of labels with which the antibody may be labelled include fluorescent labels (such as fluorescein and molecules based on fluorescein, and Cyanine3 (Cy3)), which are labels that, when excited by illumination at a relevant wavelength, will fluorescence at a different, detectable wavelength; and peroxidase labels (e.g. horseradish peroxidase), which, in the presence of hydrogen peroxide, can oxidize a chromogenic substrate or a chemiluminescent substrate, resulting in a detectable color change. After a period of incubation to allow the antibody to bind the viral substrate, typically from 15 to 60 minutes at room temperature, the sample is washed to remove unbound antibody.

Alternatively, the antibody that binds the viral substrate may be unlabeled, and after the sample is washed as stated in the preceding sentence, a second, labelled antibody that binds to the first antibody may be introduced. Examples of labels with which the second antibody may be labelled include fluorescent labels and peroxidase labels, as described above. After a period of incubation to allow the second antibody to bind the first antibody, typically from 15 to 60 minutes at room temperature, the sample is again washed to remove unbound second antibody.

In some cases, antibodies that bind to a given viral substrate are available commercially, and may be purchased and subsequently labelled. Examples of such antibodies are “Anti-SARS Nsp3 antibody -Coronavirus (ab181620)” available from Abcam (https://www.abcam.com/sars-nsp3-antibody-coronavirus-ab181620.html), which binds to a portion of the viral coat of the SARS virus; and “Anti-Coronavirus Antibody, OC-43 strain, clone 541-8F”, available from SigmaAldrich (https://www.sigmaaldrich.com/catalog/product/mm/mab9012?lang=en&region=US), which binds to a portion of the corona virus OC-43 strain. It is expected that the two aforementioned antibodies will also bind to portions of proteins of the SARS-CoV-2 coronavirus (viz. the virus responsible for the disease in humans known as COVID-19), in particular to a portion of the virus that is presented on the surface of infected cells, and thus may be used to detect infection by the SARS-CoV-2 coronavirus. Antibodies may also be available for purchase in already labelled form.

Specifically in the case of coronaviruses, such viruses have four structural proteins, and have or produce several other proteins that are not part of the viral structure. The structural proteins are the spike protein, envelope protein, membrane protein and nucelocapsid protein. Coronaviruses have several copies of these structural proteins, and it is contemplated that detection of infection by a coronavirus may be effected with an antibody or antibodies that bind to one or more of these structural proteins, whether as part of the intact structural protein or when presented as part of a cell-surface antigen. It will also be appreciated that infection by certain coronaviruses, including SARS-CoV-2 and SARS-CoV, also induces production of viral proteins in the infected cells, which may then be present on the cell surface and/or within the cell, and therefore it is possible to detect these proteins expressed in infected cells, using antibodies specific to the proteins in question. In the case in which one or more of the viral proteins are present within the cell, the sample will need to be contacted with a permeabilization agent prior to being contacted with the antibody or antibodies that binds to the protein(s) of interest, to facilitate penetration of the antibody (antibodies) into the cell.

In a variation of the process described above, in the case of a virus having different proteins, detection may be effected using two different antibodies that bind to different viral proteins (whether those proteins are part of the virus, present within the cell, or on the cell surface), whether as part of the intact protein or when presented as part of a cell-surface antigen, and by labeling the two different antibodies with respective labels that produce different signals. For example, in the case of a coronavirus, a first antibody that binds to e.g. the spike protein (when present on the virus surface or on the surface of an infected cell) or to a portion of the spike protein when presented as part of a cell-surface antigen, may be employed. This first antibody may itself be labelled with a first label, as described above, or it may be unlabeled, in which case a second antibody labelled with a first label is used to bind to the first antibody. A third antibody, which binds to e.g. the nucleocapsid protein (when present as part of the virus, in an infected cell, or on the surface of an infected cell) or to a portion of the nucleocapsid protein when presented as part of a cell-surface antigen, is also used, and contacted with the sample simultaneously or sequentially with contacting of the sample with the first antibody. The third antibody may be labelled with a second label that is different from the first label, or it may be unlabeled, in which case a fourth antibody labelled with a second label that is different from the first label is used to bind to the third antibody. The fourth antibody should not bind the first or second antibodies. In order to avoid binding of the fourth antibody to the first or second antibody, it may be preferable that the third antibody be derived from a different mammalian source than the first and second antibodies. As noted above, in the case in which one or both viral proteins are present within the cell, the sample will need to be contacted with a permeabilization agent prior to being contacted with the antibody or antibodies that bind to the protein(s) of interest, to facilitate penetration of the antibody (antibodies) into the cell. It is sufficient to contact the sample with permeabilization agent once after or simultaneously with fixation of the cells in the sample. It will be appreciated that contacting the sample simultaneously with fixation agent and a permeabilization agent, and using a single wash to remove both agents, reduces the total time to conduct the test. The first and second labels may, for example, fluoresce at different excitation frequencies and/or produce light at different respective frequencies in response to excitation. If the virus of interest is present in the sample, the use of two labels for two different viral proteins facilitates the detection of two different signals in the image(s) obtained. This facilitates improved accuracy of the test. It will be appreciated that in the situation in which one or both of the first and third antibodies is unlabeled, it is preferable that the first and third antibodies be derived from different sources, e.g. one from mouse and one from rabbit, so that the labelled second antibody specifically binds the first antibody, and/or the labelled fourth antibody specifically binds the third antibody. It will also be appreciated that a fifth antibody that binds yet a third viral protein or portion thereof, either as part of the intact protein or when presented as part of a cell-surface antigen, e.g. the envelope protein of a coronavirus, may be used, and that this fifth antibody may be labelled with a third label or that a sixth, labelled antibody that binds the fifth antibody may be used, in which case the fifth antibody should be derived from a different source than the first and third antibodies. It will also be appreciated that in the interest of reducing the time it takes to test for the presence of viral protein, certain steps may be conducted simultaneously, e.g. the contacting with fixation agent and permeabilization agent may be done simultaneously, as described above, and/or the contacting with the first and third antibodies may be done simultaneously, and/or the contacting with the second and fourth antibodies may be done simultaneously. This eliminates both a contacting and/or incubation step, as well eliminates as a wash step. Similarly, using labeled first and third antibodies will reduce the total time required, since this obviates the need to contact, incubate and wash with the second and fourth antibodies.

In the event that an antibody that binds to a viral antigen of interest is not commercially available, and it is necessary to produce one or more such antibodies, the production of antibodies to viral proteins is well-known in the art. See, for example, Yokoyama et al., Current Protocols in Immunology, 2006, Supplement 74, which is incorporated herein by reference; Greenfield, “Generating Monoclonal Antibodies”, in Antibodies: A Laboratory Manual, 2nd Edition, Spring Harbor Press 2014; and Ossipow et al., “Monoclonal Antibodies Methods and Protocols Second Edition”, Humana Press, Spring Science+Business Media, New York 2014, the contents of which are incorporated herein by reference. While in some embodiments monoclonal antibodies may be used as the first and third antibodies, respectively, in other embodiments polyclonal antibodies may be used, provided that the first polyclonal antibody binds a first viral protein and the other polyclonal antibody binds the second viral protein.

The labelling of antibodies is likewise well-known in the art, and kits for antibody labelling are commercially available, e.g. from Thermo-Fischer Scientific (https://www.thermofisher.com/il/en/home/life-science/antibodies/antibody-labeling.html).

In one embodiment, when one or more labelled antibodies that bind to a viral substrate are used, each labelled antibody may be provided as part of a kit in lyophilized form, i.e. as a dry powder, and each such powder may be mixed with a buffer solution, such as phosphate buffer solution, which may also be provided as part of a kit, to form one or more labelled-antibody-containing solutions, and the labelled-antibody-containing solution(s) may then be brought into contact with the sample. Any buffered solutions used may themselves be prepared on-site, with the ingredients of the buffer being provided in dry form, and water being mixed with the buffer ingredients to form the buffer solution. The same or a different buffer solution may be used for washing away unbound antibody. Similarly, if one or more pairs of labelled and unlabeled antibody are used, all the antibodies may be provided in lyophilized form. It may be helpful to include an anti-bacterial material, such as a small amount of sodium azide, in the buffer.

In some embodiments, it may be desirable to contact the sample with a non-antibody protein prior to contacting the sample with antibody, in order to reduce non-specific binding of antibody to the sample. Such a protein, sometimes referred to as a “blocking protein”, may be provided in lyophilized form to be mixed with buffer to facilitate contacting with the sample, e.g. as a part of a kit. It may also be desirable to utilize a permeabilization agent prior to addition of the antibody. The contacting with the blocking protein may be effected simultaneously with or after fixataion of the cells in the sample and/or before, after or simultaneously with the contacting of the sample with a permeabilization agent.

As noted, if one or more of the viral proteins are present within the cell, the sample will need to be contacted with a permeabilization agent, also sometimes referred to as a penetration agent, prior to being contacted with the antibody that binds to the protein of interest within the cell, to facilitate penetration of the antibody into the cell. Such permeabilization agents are generally divided into two groups, viz. organic solvents such as methanol and acetone, and detergents such as saponin, Triton X-100 and Tween-20. The permeabilization agent will then be washed away with buffer prior to bringing the antibody into contact with the sample. The permeabilization agent may also be supplied as part of a kit, either in dry form, optionally with buffer ingredients, to which water or buffer solution is added, or in solution form, optionally with buffer.

Following the binding of labelled antibody or, if more than one labelled antibody is used, all labelled antibodies, and washing away of excess antibody, the conditions of the sample in the sample vessel are then adjusted to elicit a signal from the label(s) on the labelled antibody or antibodies that may be present. For example, in the case of a fluorescent label, fluorescence is elicited by irradiating the fluorescent label with light of the appropriate wavelength to induce fluorescence. In the case of a peroxidase label, hydrogen peroxide and a chromogenic substrate or a chemiluminescent substrate for the label are introduced, eliciting a change in color.

During the time period that the label, if present, will produce the signal, one or more images of the sample are then captured, in order to facilitate analysis and determine whether or not the label, and thus labelled antibody and therefore the viral substrate, is present in the sample. If two antibodies that bind to two different viral proteins or portions thereof when presented as part of a cell-surface antigen are used, it possible that multiple signals may be produced from a single set of signal-inducing conditions, or that two different sets of signal-inducing conditions may be employed simultaneously. For example, if two different fluorescent labels are used, it may be possible to irradiate so as to obtain fluorescence from both labels simultaneously; or, if one label is a fluorescent label and the other a peroxidase label, it may be possible to generate fluorescence while the peroxidase is active on its substrate. In such cases, capturing a single image or group of images under the signal-inducing conditions may be sufficient. However, it is also possible that different sets of signal-inducing conditions will be required in sequence, in which case a series of images may be captured, at least one image, and preferably a plurality of images, in response to each set of signal-inducing conditions. For example, irradiation with light that induces fluorescence in one label may be used and a first set of images captured, followed by irradiation with light that induces fluorescence in the other label, and another set of images captured. The images captured in response to one set of signal-inducting conditions may then be compared with the images captured in response to the other set of signal-inducting conditions, or a composite image from image(s) captured in response both sets of signal-inducting conditions may be generated. The presence of a signal or signals in the images will normally be indicative of infection of cells in the sample by the virus.

In some embodiments, the image generation and acquisition will be conducted using a dedicated fluorescence microscope or scanning device, such as the Hermes system, available from Idea Biomedical Ltd., Rehovot, Israel (https://idea-bio.com/products/wiscan-hermes/). In some embodiments, the image generation and acquisition will be conducted using a common cellular telephone device of the variety that is equipped with a camera and a light-emitting diode (LED) as a means of illumination, and is capable of transmitting images stored in the device, such as an Apple iPhone or a Samsung Galaxy phone, or with a common tablet computer, such as an Apple iPad or Microsoft Surface, which is similarly equipped with a camera and illumination means such as a LED, and capable of transmitting images stored in the device. The optics of most cellphone cameras and tablet computers are insufficient for obtaining images of sufficiently high resolution for present purposes, e.g. about 0.8 micrometers resolution is required for a sufficiently resolved image if detailed cell by cell analysis is to be applied. Consequently, it may be necessary to retro-fit the device with optics that increase the magnification of the camera, so that the resulting image shows a smaller area but at higher resolution than in the absence of the optics. An example of such is the TINYSCOPE Mobile Microscope, available at https://www.amazon.com/TINYSCOPE-Microscope-Magnification-Portable-Batteries/dp/B07XSZP6YY/ref=pdsbs__421t 2/135-8243463-3310041? encoding=UTF8&pd rd i=-BD7XSZP6YY&pd rd r=55f430e4-e2ec-437c-9be2-a54a90c2c756&pd rd w=dfwCX&pd rd wg=D55gt&pf rd p=5cfcfe89-300f-47d2-b1ad-a4e27203a02a&pf rdr=PDQ8W8XCG3C7945K7EZ1&psc=1&psc&refRID=PDQ8W8XCG3C7945K7EZ1. The retro-fitted optics may be mounted directly to the cellphone or tablet computer, or they may be mounted in a separate platform on which the cellphone or tablet computer may be placed.

Most cellular phones sold today, and many tablet computers, are equipped with a light-emitting diode (LED) that may be used to illuminate an area, by providing a continuous output of light. Such devices are usually configured so that that LED, or one more other LEDs, may also function to provide a brief flash of light when photographs are taken in dim lighting conditions. The LED(s) provide white light across substantially the entire visible spectrum, and may be used both to excite the fluorescent label and to illuminate the sample in the sample vessel. In order to obtain an image of a fluorescing antibody label, which fluoresces at a particular wavelength but which may provide a weak signal that is overwhelmed by the reflection of illuminating/exciting flash used, it may be necessary to utilize a filter or filters that allows light at the fluorescent wavelength(s) to pass through the camera lens but filters out enough light at other wavelengths to facilitate acceptable detection of fluorescence. It may be helpful to capture multiple images of the sample’s response to the excitation. It also may be important to acquire images with and without the filters (transmission image in addition to the fluorescence image). In some cases, it may also be helpful or necessary to filter the excitation light; in such cases the excitation light filter may be different than the filter for the imaging light. In some cases, it may be necessary to let the excitation light fade prior to capturing the image of the fluorescence. Software to enable the cellular telephone device or tablet computer to illuminate, and then capture an image after the illumination has been discontinued, may be provided, or the device may be equipped (by retro-fitting directly on the device, or by mounting in a separate platform on which the device may be placed) with a filter on the excitation light that after a certain amount of exposure becomes opaque and blocks the excitation light. If a labeling method other than fluorescence is used, the LEDs may be used as necessary to illuminate the sample and facilitate image capture.

Once at least one image has been captured and, optionally, stored in the device’s memory, it may then be transmitted (e.g. via a cable, through cellular phone transmission or a WiFi network or over the internet) to a remotely located computer for analysis, to determine if the degree of fluorescence or other change, e.g. change in cellular morphological features, which is detected are indicative of the presence of the virus of interest. In some embodiments, software installed on the recording device, e.g. the designated microscope, cellular phone or tablet computer, will provide the analysis, without the need for transmission to a remote location.

Analysis of the images may be based on the detection of fluorescence intensity at the wavelength of the labeled antibody. Bulbs of intensity may be detected by initially conducting a small amount of smoothing of the image, followed by background subtraction, and then manipulation using a watershed algorithm. Bulbs of intensity below a set threshold will be ignored. The total number of pixels presenting fluorescence above the threshold in the bulbs detected using the watershed algorithm may then be counted. If this number is above a threshold value, the system will report a positive answer. If labelled antibodies that bind to different viral proteins are used, then the threshold may be set on the basis of a combination of pixels pertaining to each signal. It will be appreciated that the analysis may employ artificial intelligence algorithms that detect desired objects and detect morphological features in the sample using dedicated algorithms and parameters.

Optionally, prior to capturing images, the sample may be stained using one or more materials that stain cell nuclei or stain the cell membrane. An example of a material that stains cell nuclei is 4′,6-Diamidine-2′-phenylindole dihydrochloride, also referred to as DAPI; an example of material that stains the cell membrane is the CellMask Deep Red Plasma Membrane Stain, ThermoFisher catalog no. C10046. The use of such stains helps identify individual cells or cell nuclei in the images, and can be useful in both automated and manual image analysis, for example to determine the number of infected cells and total number of cells in a sample, or to determine viral protein density in a single infected cell.

It will be appreciated that even though some time is required to allow for antibody conjugation, typically 15-60 minutes, the time required to test an individual for the presence of a virus is decreased relative to standard virus detection methods presently in use, as there is no need to culture cells or perform PCR. Additionally, since an individual may provide the sample and obtain the image himself, there is no need to wait for a sample to be sent to a lab for culturing or PCR. If an individual is suspected of being infected with a highly contagious virus, the individual may be provided with a test kit (an example of such is discussed below) and the necessary data for diagnosis obtained and transmitted at home or in a quarantine area, minimizing the exposure of others to the individual and the possibly virus-infected sample. Alternatively or additionally, if it is desired to test many patients in a short amount of time, a plurality of samples from different individuals may obtained, placed in separate sample vessels, e.g. the wells of a multiwell plate, with identifying information being provided for each sample, e.g. in the form a small barcode uniquely identifying the sample in each well that contains a sample. A small number of images will then be sufficient for analysis of the entire plate. This variation provides the option of increasing the throughput of testing. For example, a testing center could be set up in the field, in which samples from a large number of individuals could be collected and multiple imaging devices such as the Hermes system could be used in parallel to analyze the samples, so that thousands of samples could be analyzed over the course of a day in a single testing center. As noted, the ability to contact a sample with antibody soon after sampling, without the need to first dilute, centrifuge, grow or otherwise manipulate the cells, greatly speeds the process relative to presently conventional techniques for the detection of viral infection in mammalian cells.

In view of the foregoing, it will be appreciated that there is also provided, in accordance with an embodiment of the present invention, a method for testing a mammalian patient for the presence of a coronavirus in the patient, the method involving:
1. obtaining a sample of mucus from a patient
2. transferring the sample which has been obtained from a patient to a sample vessel, the sample vessel being substantially free of cells other than those in the sample, wherein between the obtaining of the sample and the transferring of the sample to the sample vessel the sample is not (a) incubated with other cells, (b) diluted, including diluted by contacted with a fixing solution; (c) frozen or (d) centrifuged;
   2A. fixing the cells of the sample by contacting them with a fixing solution for a time sufficient to fix the cells in the sample vessel, and thereafter conducting an initial washing of the fixed cells with a washing solution;
   2B. optionally, contacting the sample with a blocking protein, and thereafter washing the sample;
   2C. optionally, contacting the sample in the vessel with a permeabilization agent, and thereafter washing the sample;wherein said contacting the sample with a permeabilization agent, if conducted, is conducted simultaneously with or subsequent to said contacting with a fixing solution, and wherein said contacting with a blocking protein, if conducted, is conducted simultaneously with or subsequent to said contacting with a fixing solution; and wherein, if said contacting the sample with a permeabilization agent is conducted simultaneously with said contacting with a fixing solution, said conducting an initial washing and said thereafter washing are done as a single washing step; and wherein, if said contacting the sample with a blocking protein is conducted simultaneously with said contacting with a fixing solution, said conducting an initial washing and said thereafter washing are done as a single washing step;
3. contacting the sample in the sample vessel with a first antibody known to bind a first viral substrate, the first viral substrate being at least one of (i) an intact first coronavirus structural protein and (ii) a portion of a first coronavirus structural protein when presented as part of a cell-surface antigen,
4. after a first incubation period to allow binding of the first antibody to the viral substrate, conducting a first washing of the sample in the sample vessel
5. if the first antibody is labeled with a first label, skipping to 6; if the first antibody is not labeled with a first label, after the first washing contacting the first antibody with a second antibody that binds the first antibody, the second antibody being labeled with a first label, and after a second incubation period to allow binding of the second antibody to the first antibody, conducting a second washing the sample in the sample vessel;
6. inducing conditions which will cause the first label, if present, to produce a first signal
7. during the time period in which the first signal would be produced if the first label is present in the sample, capturing at least one first image of the sample in the sample vessel,
8. at least one of (a) sending the at least one first image to a remote analysis station, and (b) analyzing the at least one first image to determine the presence or absence of the first signal, wherein the presence of the first signal indicates presence of the virus in the sample.

In some embodiments, the method further comprises:
3A. contacting the sample in the sample vessel with a third antibody known to bind a second viral substrate, the second viral substrate being at least one of (i) an intact second coronavirus structural protein and (ii) a portion of a second coronavirus structural protein when presented as part of a cell-surface antigen, the second corona virus structural protein being different from the first coronavirus structural protein;
4A. after a third incubation period to allow binding of the third antibody to the second viral substrate, conducting a third washing of the sample in the sample vessel;
5A. if the third antibody is labeled with a second label, skipping to 6A; if the third antibody is not labeled with a second label, after the third washing contacting the third antibody with a fourth antibody that binds the third antibody, the fourth antibody being labeled with a second label, and after a fourth incubation period to allow binding of the fourth antibody to the third antibody, conducting a fourth washing of the sample in the sample vessel;
6A. inducing conditions which will cause the second label, if present, to produce a signal
7A. during the time period in which the second signal would be produced if the second label is present in the sample, capturing at least one second image of the sample in the sample vessel,
8A. at least one of (a) sending the at least one second image to a remote analysis station, and (b) analyzing the at least one image to determine the presence or absence of the signal, wherein the presence of the signal indicates presence of the virus in the sample.

In some embodiments, steps 3 and 3A are carried out simultaneously, the first incubation period and the third incubation period are a single incubation period, and the first and third washing are a single washing. In some embodiments, both the first antibody and the third antibody are labeled with the first label and second label respectively.

In some embodiments, the second incubation period and the fourth incubation period are a single incubation period, and the second and fourth washing are a single washing. In some embodiments, both the second antibody and the fourth antibody are labeled with the first label and second label respectively.

In some embodiments, 7 and 7A are carried out simultaneously, and the at least one first image and the at least one second image are the same at least one image; and 8 and 8A are carried out simultaneously. In such embodiments, the presence of both the first and second signals in the image is a better indication of the presence of the virus than the presence of only the first signal or only the second signal.

In order to facilitate testing, there is also provided, in accordance with an embodiment of the invention, a kit, the kit containing a first antibody that binds to a first viral substrate of interest; a first fluorescent or other label, which may be provided covalently bonded to the first antibody that binds the viral substrate, or may be covalently bonded to a second antibody that binds to the first antibody; optionally, a third antibody that binds to a second viral substrate of interest; if the third antibody is present, a second fluorescent or other label, which may be provided covalently bonded to the third antibody that binds the second viral substrate, or may be covalently bonded to a fourth antibody which is provided and that binds to the third antibody; and a sample vessel. The fourth antibody, if present, should not bind the first or second antibodies. Thus, it may be preferable, if the first and third antibodies are not themselves labeled, that the first and second antibodies are derived from a different mammalian source than the third antibody. Each antibody may independently be provided in lyophilized form, preferably in a vessel into which a defined amount of buffer solution is added and a mixed, or the vessel may contain buffer ingredients in addition to lyophilized antibody, in which case a defined amount of water is added to the vessel and mixed. Optionally, the kit also contains an optical apparatus for increasing the magnification of a cellphone camera or tablet computer camera; a light filter that allows light at the fluorescent wavelength to pass through but filters out enough light at other wavelengths to facilitate acceptable detection of fluorescence by the cellphone camera or tablet computer camera; and optionally a light filter that filters the excitation light. Optionally, the kit may contain a sampling apparatus, such as a cotton swab. The kit may also contain one or more buffer solutions for preparing one or more antibody solutions, and for washing excess antibody, or the kit may be provided with buffer ingredients and a mixing vessel in which the buffer ingredients and water are mixed to prepare a buffer solution. The kit may also contain a solution of fixation agent, or it may contain dry fixation agent in a vessel to which buffer solution is added and mixed. The kit may also contain a solution of permeabilization agent, or it may contain dry permeabilization agent to which buffer solution is added and mixed. The kit may also contain directions for its use, including, for example, how to obtain a sample and place it in the sample vessel, how to place the optics and filter on the camera, how to obtain an image, and an internet address to which the image(s) obtained may be transmitted for analysis, and/or from which may be downloaded one or more of (a) detailed instructions on how to use the kit and upload images, (b) software to be installed to help use the kit, (c) an analysis and report of results. An example of such a kit is shown at pages 9 and 10 of the attached Appendix B. The kit shown is provided as a rectangularly-shaped box mount having an upper surface and side surfaces, but without a bottom surface. The kit shown is provided with four sterile cotton swabs, each in its own tube which is stored within the rectangularly-shaped mount. Also provided in the kit shown is a glass slide, which initially is stored in a slide holder portion of the mount, and has specific locations indicated for the placement of each the samples to be collected using the swabs. Buffer solutions, fixation solutions, and containers containing antibody to which buffer solution may be added are labelled “compound A”, “compound B”, “compound C” and “compound D”, respectively. When the cap of one of these containers is removed, the slide may be slid into the container, to allow contacting of the sample with an antibody, washing of the sample, etc. Mounted on the underside of the upper surface of the mount is an optical magnifier, which optionally may contain optical filtering elements. Upon completion of sample preparation, the slide may be slid back into the slide holder portion, which aligns the slide so that the sample(s) are held in alignment with the optical magnifier and an aperture in the upper surface of the mount. The cellphone or tablet computer is then placed upon the mount so that the camera and, if necessary, LED illumination is aligned with the optical magnifier and the sample(s). Software to facilitate (a) image capture and (b) image analysis and/or transmission to a remote computer may be uploaded to the cellphone or tablet computer, images acquired, and analysis of the images conducted on the device or remotely.

Also provided, in accordance with an embodiment of the invention, is a method for reducing the likelihood of a population of persons to infect others with a coronavirus, the method comprising:
a. obtaining a sample from each person in the population,
   a′. optionally, contacting one or more samples with a fixing solution;
b. transferring each sample to a sample vessel, the location of each sample in the sample vessel being substantially free of cells other than those in the respective sample, wherein between the obtaining of the sample and the transferring of the sample to the sample vessel the sample is not (a) incubated with other cells, (b) diluted with a solution other than fixing solution, (c) frozen or (d) centrifuged;
   b1. optionally, if the cells of a sample have not been previously contacted with a fixing solution, fixing the cells of the sample by contacting them with a fixing solution for a time sufficient to fix the cells in the sample vessel, and thereafter conducting an initial washing of the fixed cells with a washing solution;
   b2. optionally, contacting a sample in the sample vessel with a blocking protein, and thereafter washing the sample to remove excess blocking protein;
   b3. optionally, contacting a sample in the sample vessel with a permeabilization agent, and thereafter washing the sample to remove excess permeabilization agent;wherein two or three of steps b1, b2 and b3 may be performed in series or in parallel, and if performed in parallel a single washing step may be employed;
c. contacting each sample in its respective sample vessel with a first antibody known to bind a first viral substrate, the first viral substrate being at least one of (i) an intact first coronavirus structural protein (viz. spike protein, membrane protein, envelope protein or nucleocapsid protein of a coronavirus), and (ii) a portion of a first coronavirus structural protein when presented as part of a cell surface antigen,
d. after an incubation period to allow binding of the first antibody to the viral substrate, conducting a first washing of each sample in the sample vessel to remove excess first antibody
e. if the first antibody is not labeled with a first label, after the first washing contacting the first antibody with a second antibody that binds the first antibody, the second antibody being labeled with a first label, and after an incubation period to allow binding of the second antibody to the first antibody, washing each sample in the sample vessel to remove excess second antibody
f. inducing a first set of conditions which will cause the first label, if present, to produce a first signal
g. during the time in which the first signal would be produced if the label is present in a sample, capturing at least one first image of each sample in the sample vessel,
h. analyzing each at least one first image to determine the presence or absence of the first signal, wherein the presence of the first signal above a threshold level indicates presence of the coronavirus in the sample;
i. removing those individuals determined to have the coronavirus in their samples from the population of persons, whereby to create a reduced population of persons, and
j. optionally allowing the reduced population of persons to do at least one of the following: (1) disperse among the general public, (2) board a transportation vessel.

In some embodiments, the step of removing those individuals determined to have the coronavirus in their samples from the population of persons comprises restricting the movement of the individuals determined to have the coronavirus. In some embodiments, the restricting the movement comprises confining the individuals to one or more designated quarantine areas.

In some embodiments, the method is further characterized by:
contacting each sample with a third antibody known to bind a second viral substrate, the second viral substrate being at least one of (i) an intact second coronavirus structural protein, viz. a spike protein, membrane protein, envelope protein or nucleocapsid protein of a coronavirus, and (ii) a portion of a coronavirus structural when presented as part of a cell-surface antigen, wherein the second coronavirus structural protein a different coronavirus structural protein or derived from a different coronavirus structural protein than the first coronavirus structural protein;
the incubation period of step d also allowing binding of the third antibody to the second viral substrate, and the first washing also removing excess third antibody;
if the third antibody is not a labeled antibody, after the first washing contacting the third antibody with a fourth antibody that binds the third antibody but not the first antibody or, if present, the second antibody, the fourth antibody being labeled with a second label, and after an incubation period to allow binding of the fourth antibody to the third antibody, washing each sample to remove excess fourth antibody
simultaneously or sequentially with inducing the first set of conditions which will cause the first label, if present, to produce a signal, inducing a second set of conditions which will cause the second label, if present, to produce a signal;
during the time in which the second signal will be produced if the second label is present in a sample, capturing at least one second image of the sample in the sample vessel, which may be the same or different from the at least one first image;
analyzing the at least one first image and the at least one second image to determine the presence or absence of the first and second signals, wherein the presence of the signals above a threshold level in the same cell in a sample indicates presence of the coronavirus in the sample.

In a particular embodiment, there is no fixation of cells prior to transfer of samples to their respective sample chambers; and the first and third antibodies are labelled antibodies, obviating the need for the second and fourth antibodies.

As explained above, prior to image capture, the samples may also be stained with one or more stains that color cell nuclei and/or cell membranes.

The transportation vessel may be, for example, an airplane, a helicopter, a bus, a train, a passenger or commercial ship, a boat or a ferry.

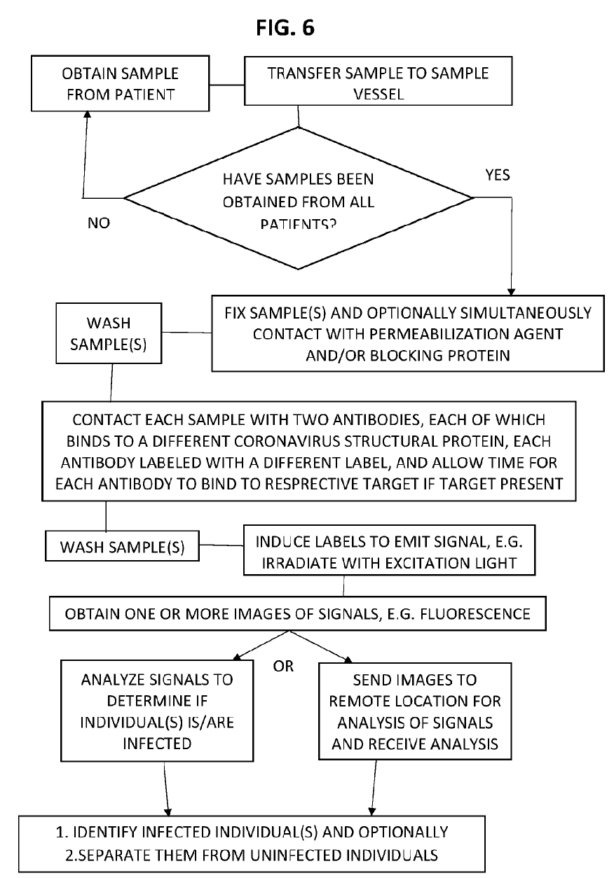

FIG. 6 shows schematically several specific embodiments of the invention. A sample to be tested for coronavirus is obtained from a patient, e.g. by obtaining mucus from the nasal passage and/or the throat with a cotton swab. This is then transferred directly to a sample vessel, e.g. a glass vessel which has been coated with polylysine, as discussed above, without first fixing, diluting, centrifuging, or incubating the sample with other cells. If there is only one individual to be tested, the sample is then contacted with fixing solution, and optionally with a permeabilization agent and/or blocking protein. If multiple individuals are to be tested, samples from those individuals are also collected, again without manipulation prior to transfer to the sample vessel, with each sample being transferred to a separate sample vessel, followed by fixation and optionally contact with a permeabilization agent and/or a blocking protein. This is followed by washing of each sample in its respectively sample vessel, e.g. with PBS buffer. Each sample is then incubated with two different antibodies that are each labeled with a different label, e.g. each labeled with a different fluorescent label. In other words, each sample is contacted with two different labeled antibodies and the antibodies are allowed to equilibrate with the samples. The two antibodies bind to a different coronavirus structural protein, e.g. on binds to the spike protein and the other to the nucleocapsid protein. After sufficient incubation, each sample is washed to remove unbound antibody. The samples are then subjected to conditions to elicit signals from the labels, if the labels are present, for example if both labels are fluorescent labels, the labels are subjected to excitation illumination. One or more images of the samples are captured. From a time-saving perspective, it is preferable if all the samples are illuminated simultaneously and images for all samples are captured simultaneously, although images for each sample may be captured separately. The images may then be analyzed on site, or they may delivered electronically or wirelessly to a remote location for analysis and then then results conveyed to the site where the images were obtained. Infected individuals may then be identified. If the samples were taken e.g. at an airport from arriving passengers, the infected individuals may be separated from the non-infected individuals.

### EXAMPLES

VeroE6 cells obtained from the Israel Institute for Biological Research and grown in Dulbecco’s Modified Eagle Medium (DMEM) with 10% fetal calf serum (FCS) at 37° C. were infected with the SARS-CoV-2 coronavirus (using minimum essential medium (MEM) with 2% fetal bovine serum (FBS)) and allowed to continue to grow for 24 hours. Some of these cells were placed in a well of a microtiter plate, and affixed thereto by contacting with 3% paraformaldehyde (PFA) for 30 minutes. The cells were then washed with phosphate buffer solution (PBS). Cells were then permeabilized using 2% Triton for 2 minutes and washed with PBS. Cells were then contacted for 40 minutes with one of two different Rabbit polyclonal antibodies that bind the spike protein of SARS-CoV-2 and then washed using PBS. The antibodies had previously been labeled with Alexa488 (ThermoFisher), which fluoresces at 520 nm when excited by light of 480 nm. Since DAPI is excited at 390 nm and emits fluorescence at 410 nm, the labeling antibody solution was also mixed with 1:100000 DAPI solution to allow simultaneous labeling of the nuclei and the sample again washed with PBS. The sample was then imaged using the WiScan Hermes machine, using LED light to excite at 390 nm and 488 nm and obtain the fluorescence images of the DAPI and the Alexa488 respectively. Images of the DAPI fluorescence show the locations of the cell nuclei, while images of the antiSpike antibody labeled with the Alexa488 show the cell compartments that were infected with the SARS2-CoV. The DAPI and Alexa488 images were analyzed separately to identify the relevant structures in each. A composite image was made for visualization purposes.

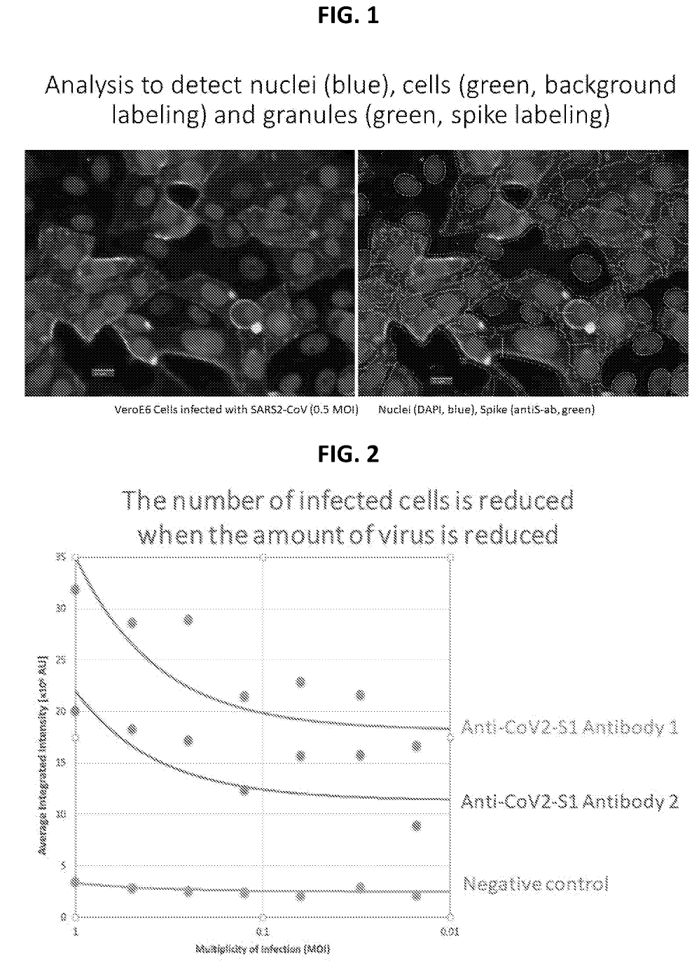

The images were analyzed using the Athena software program provided with the Hermes scanner. This program automatically identified individual cells and fluorescence from the labels, and calculated the fluorescence intensity for each cell. Cells exhibiting intensity above a pre-determined cutoff were identified by the program as infected with the virus. See FIG. 1, in which the nuclei are shown in blue, and cellular components infected by the virus are shown in green. There are granular structures that were detected in the cells, which may be aggregates of spike protein. Only infected cells (containing spike protein) were labeled in green, while nuclei were labeled in both infected and uninfected cells. The outline of the analyzed objects are shown in the image on the right, identifying the analyzed features (nuclei, cell outline, granular features).

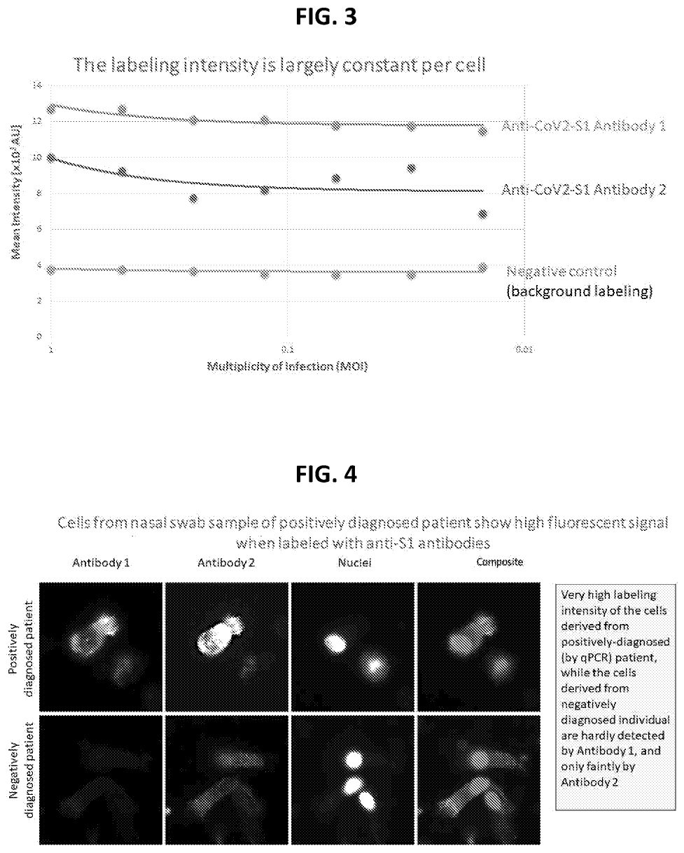

The program also calculated the average signal for those cells in which fluorescence was detected. Although, as expected, when a lower concentration of virus was used to infect the cells, fewer cells became infected during the incubation period, it was found that the signal intensity per infected cell did not change appreciably between infected cells, even in samples that had few infected cells. These observations are shown FIGS. 2 and 3, respectively, in which “multiplicity of infection” refers to the concentration of virus used to infect the cell sample. FIG. 2 shows that a lower multiplicity of infection (MOI, a unit to measure the amount of viruses used to infect the sample) leads to a lower overall signal intensity in a given sample; FIG. 3 shows that signal intensity per infected cell does not change appreciably with MOI:

It another experiment, the procedure described above was repeated, except that two or three types of antibodies to the spike protein, each type of antibody labeled with a different label, were used for detection. Each label fluoresced at a different frequency. The software identified infected cells on the basis of the presence of both colors or all three colors above a threshold value.

In another experiment, patients’ nasal passages were swabbed for cells, and the swabs were then dipped in 3% PFA, and then wiped in tube which was then washed and frozen. After thawing, the tube with the sample was centrifuged, and the pellet of cells obtained was removed and placed on a glass microscope slide. Cells were then incubated with two different antibodies, one a polyclonal antibody having a first fluorescent label and the other a monoclonal antibody having a second fluorescent label. Both antibodies bind to the spike protein of SARS-CoV-2; the nuclei of the cells were also stained. Samples were then irradiated with excitation radiation to elicit fluorescence, and images captured, and a composite image generated. Images from the samples of control patients -three patients who had been determined to be COVID-19 negative on the basis of PCR testing -showed very weak signals from both antibodies, whereas three patients who had been determined positive for COVID-19 on the basis of PCR testing had strong signals for both antibodies. See FIG. 4.

The above procedure may also be conducted, but instead of using two different antibodies to the same viral protein, two different antibodies to two different coronavirus structural proteins may be employed, with analysis conducted as described above.

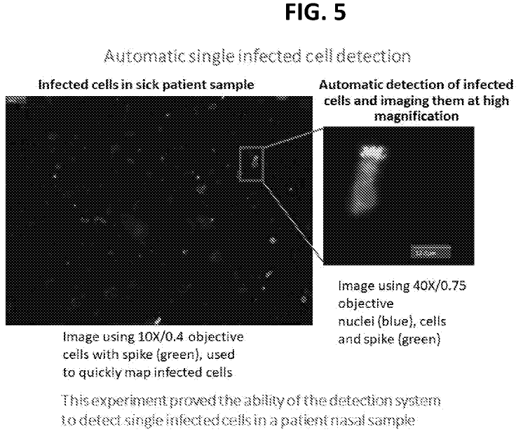

In another experiment, a nose swab from a confirmed COVID-19 positive patient was fixed by dipping the swab in 3% PFA and then the swab was wiped on the bottom of a well of a multiwell plate to transfer cells to the plate. Cells were then incubated with a labeled polyclonal antibody, and the nuclei stained. The sample was then irradiated with excitation radiation to elicit fluorescence, and images captured at low resolution, in order to identify the position in the plate in which the sample was located. At this low magnification, the Athena program mapped the locations of individual cells in the sample on the basis of both nuclear staining and antibody signal. The cell was then imaged again at higher magnification (40×), which enabled identification of structural features within the cell, as well as the distribution of signal intensity within the cell. See FIG. 5. This experiment proved the ability of this methodology to detect single fluorescence cell in a swab nasal sample.

It will be appreciated that despite the prevalence of color drawings and photographs in the scientific literature and the ease of presentation of such in electronic format, PCT rules remain mired in the 19th century and still do not permit the filing of color drawings or photographs, and the USPTO only allows color drawings or photographs pursuant to a petition, and such color drawings or photographs cannot be submitted via the USPTO electronic filing system. Therefore FIGS. 1, 2, 3, 4 and 5 are being filed with this PCT application as grayscale photographs. However, the original photographs are in color and, for the purpose of making them publicly available, have been uploaded to a publicly-available picture sharing service, Shutterfly, and can be accessed over the internet by anyone using the link https://link.shutterfly.com/vPpSf45g4db; the link is being shared for the first time with the filing of this patent application, and the color photographs are incorporated herein by reference. The color photographs are also being filed with WIPO in “pre-conversion format”.

In this description, where it stated that something is “at least one of (a) and (b)”, this means that the something may be a, may be b, or may be a combination of a and b. Similarly, “at least one of (a), (b) and (c)” means the thing may be a, or may be b, or may be c, or may be a combination of a and b, or may be a combination of a and c, or may be a combination of b and c, or may be a combination of a, b and c. Where it is stated that something may be “a and/or b”, this means the thing may a, may be b, or may be a combination of a and b.

The sequence of the SARS-Cov-2 virus from the genome NC_045512.2 is available at https://www.ncbi.nlm.nih.gov/nuccore/NC 045512.2 The sequence of the spike protein for this virus is:

MFVFLVLLPLVSSQCVNLTTRTQLPPAYTNSFTRGVYYPDKVFRSSVLHSTQDLFLPFFSNVTWFHAIHV SGTNGTKRFDNPVLPFNDGVYFASTEKSNIIRGWIFGTTLDSKTQSLLIVNNATNVVIKVCEFQFCNDPF LGVYYHKNNKSWMESEFRVYSSANNCTFEYVSQPFLMDLEGKQGNFKNLREFVFKNIDGYFKIYSKHTPI NLVRDLPQGFSALEPLVDLPIGINITRFQTLLALHRSYLTPGDSSSGWTAGAAAYYVGYLQPRTFLLKYN ENGTITDAVDCALDPLSETKCTLKSFTVEKGIYQTSNFRVQPTESIVRFPNITNLCPFGEVFNATRFASV YAWNRKRISNCVADYSVLYNSASFSTFKCYGVSPTKLNDLCFTNVYADSFVIRGDEVRQIAPGQTGKIAD YNYKLPDDFTGCVIAWNSNNLDSKVGGNYNYLYRLFRKSNLKPFERDISTEIYQAGSTPCNGVEGFNCYF PLQSYGFQPTNGVGYQPYRVVVLSFELLHAPATVCGPKKSTNLVKNKCVNFNFNGLTGTGVLTESNKKFL PFQQFGRDIADTTDAVRDPQTLEILDITPCSFGGVSVITPGTNTSNQVAVLYQDVNCTEVPVAIHADQLT PTWRVYSTGSNVFQTRAGCLIGAEHVNNSYECDIPIGAGICASYQTQTNSPRRARSVASQSIIAYTMSLG AENSVAYSNNSIAIPTNFTISVTTEILPVSMTKTSVDCTMYICGDSTECSNLLLQYGSFCTQLNRALTGI AVEQDKNTQEVFAQVKQIYKTPPIKDFGGFNFSQILPDPSKPSKRSFIEDLLFNKVTLADAGFIKQYGDC LGDIAARDLICAQKFNGLTVLPPLLTDEMIAQYTSALLAGTITSGWTFGAGAALQIPFAMQMAYRFNGIG VTQNVLYENQKLIANQFNSAIGKIQDSLSSTASALGKLQDVVNQNAQALNTLVKQLSSNFGAISSVLNDI LSRLDKVEAEVQIDRLITGRLQSLQTYVTQQLIRAAEIRASANLAATKMSECVLGQSKRVDFCGKGYHLM SFPQSAPHGVVFLHVTYVPAQEKNFTTAPAICHDGKAHFPREGVFVSNGTHWFVTQRNFYEPQIITTDNT FVSGNCDVVIGIVNNTVYDPLQPELDSFKEELDKYFKNHTSPDVDLGDISGINASVVNIQKEIDRLNEVA KNLNESLIDLQELGKYEQYIKWPWYIWLGFIAGLIAIVMVTIMLCCMTSCCSCLKGCCSCGSCCKFDEDD SEPVLKGVKLHYT

The sequence of the nucleocapsid protein for this virus is:

MSDNGPQNQRNAPRITFGGPSDSTGSNQNGERSGARSKQRRPQGLPNNTASWFTALTQHGKEDLKFPRGQ GVPINTNSSPDDQIGYYRRATRRIRGGDGKMKDLSPRWYFYYLGTGPEAGLPYGANKDGIIWVATEGALN TPKDHIGTRNPANNAAIVLQLPQGTTLPKGFYAEGSRGGSQASSRSSSRSRNSSRNSTPGSSRGTSPARM AGNGGDAALALLLLDRLNQLESKMSGKGQQQQGQTVTKKSAAEASKKPRQKRTATKAYNVTQAFGRRGPE QTQGNFGDQELIRQGTDYKHWPQIAQFAPSASAFFGMSRIGMEVTPSGTWLTYTGAIKLDDKDPNFKDQV ILLNKHIDAYKTFPPTEPKKDKKKKADETQALPQRQKKQQTVTLLPAADLDDFSKQLQQSMSSADSTQA

The sequence of the envelope protein for this virus is:

MYSFVSEETGTLIVNSVLLFLAFVVFLLVTLAILTALRLCAYCCNIVNVSLVKPSFYVYSRVKNLNSSRV PDLLV

The sequence of the membrane protein for this virus is:

MADSNGTITVEELKKLLEQWNLVIGFLFLTWICLLQFAYANRNRFLYIIKLIFLWLLWPVTLACFVLAAV YRINWITGGIAIAMACLVGLMWLSYFIASFRLFARTRSMWSFNPETNILLNVPLHGTILTRPLLESELVI GAVILRGHLRIAGHHLGRCDIKDLPKEITVATSRTLSYYKLGASQRVAGDSGFAAYSRYRIGNYKLNTDH SSSSDN IALL VQ

Variants of this virus have been observed since this virus was first sequenced, some of which are more infectious than the original virus. For example, the spike protein of the so-called British variant, B.1.1.7 major version, is:

MFVFLVLLPLVSSQCVNLTTRTQLPPAYTNSFTRGVYYPDKVFRSSVLHSTQDLFLPFFSNVTWFHAISGTNGTKRF DNPVLPFNDGVYFASTEKSNIIRGWIFGTTLDSKTQSLLIVNNATNVVIKVCEFQFCNDPFLGVYHKNNKSWMESEF RVYSSANNCTFEYVSQPFLMDLEGKQGNFKNLREFVFKNIDGYFKIYSKHTPINLVRDLPQGFSALEPLVDLPIGINIT RFQTLLALHRSYLTPGDSSSGWTAGAAAYYVGYLQPRTFLLKYNENGTITDAVDCALDPLSETKCTLKSFTVEKGIYQ. TSNFRVQPTESIVRFPNITNLCPFGEVFNATRFASVYAWNRKRISNCVADYSVLYNSASFSTFKCYGVSPTKLNDLCF TNVYADSFVIRGDEVRQIAPGQTGKIADYNYKLPDDFTGCVIAWNSNNLDSKVGGNYNYLYRLFRKSNLKPFERDIS TEIYQAGSTPCNGVEGFNCYFPLQSYGFQPTYGVGYQPYRVVVLSFELLHAPATVCGPKKSTNLVKNKCVNFNFNG LTGTGVLTESNKKFLPFQQFGRDIDDTTDAVRDPQTLEILDITPCSFGGVSVITPGTNTSNQVAVLYQGVNCTEVPV AIHADQLTPTWRVYSTGSNVFQTRAGCLIGAEHVNNSYECDIPIGAGICASYQTQTNSHRRARSVASQSIIAYTMSL GAENSVAYSNNSIAIPINFTISVTTEILPVSMTKTSVDCTMYICGDSTECSNLLLQYGSFCTQLNRALTGIAVEQDKNT QEVFAQVKQIYKTPPIKDFGGFNFSQILPDPSKPSKRSFIEDLLFNKVTLADAGFIKQYGDCLGDIAARDLICAQKFNG LTVLPPLLTDEMIAQYTSALLAGTITSGWTFGAGAALQIPFAMQMAYRFNGIGVTQNVLYENQKLIANQFNSAIGKI QDSLSSTASALGKLQDVVNQNAQALNTLVKQLSSNFGAISSVLNDILARLDKVEAEVQIDRLITGRLQSLQTYVTQQ LIRAAEIRASANLAATKMSECVLGQSKRVDFCGKGYHLMSFPQSAPHGVVFLHVTYVPAQEKNFTTAPAICHDGKA HFPREGVFVSNGTHWFVTQRNFYEPQIITTHNTFVSGNCDVVIGIVNNTVYDPLQPELDSFKEELDKYFKNHTSPDV DLGDISGINASVVNIQKEIDRLNEVAKNLNESLIDLQELGKYEQYIKWPWYIWLGFIAGLIAIVMVTIMLCCMTSCCS CLKGCCSCGSCCKFDEDDSEPVLKGVKLHYT

In the present disclosure, when reference is made to SARS-Cov-2, the intent is to refer to the NC_045512.2 version, and to any variants thereof that cause COVID-19 in humans. The spike, nucelocapsid, envelope and membrane proteins of such variants are expected to have at least 90% amino acid homology to the spike, nucelocapsid, envelope and membrane proteins of the NC_045512.2 version, viz. at least 90%, at least 91%, at least 92%, at least 93%, at least 94%, at least 95%, at least 96%, at least 97%, at least 98% or at least 99% homology to the the spike, nucelocapsid, envelope and membrane proteins of the NC_045512.2 version.

### APPENDIX B

### Viral Detection Kit and Application

### APPENDIX B

### The Need

The rapid spread of the corona virus is initially treated by isolating groups of people exposed to the virus carriers or infected.

Existing equipment and testing methods do not enable testing groups of thousands of people at once. This poses a threat to a proper working economy in Israel and the world.

[image]

### APPENDIX B

### The Proposed Solution

Idea Bio-Medical is developing a test kit that will allow hundreds of tests to be performed at a checkpoint and / or self-test at home.

This will allow tens of thousands of tests per day to be performed in Israel in the geographical distribution of several checkpoints in hospitals, airports, and military facilities.

The test concept is based on implementation of the company’s technology which enables automatic image and analysis of biological samples.

[image]

### APPENDIXB

The sample for the detection process is a throat swab smeared on a slide or a multi-well plate.

The throat swab contains epithelial cells, which will be infected by the respiratory system virus if present.

The infected cells present viral epitope on their membrane. These epitopes will be detected by a specific antibody which will be labeled fluorescently.

For the SARS-CoV-2 the antibody against membrane spike epitope developed for the SARS-CoV identified in 2003, should efficiently bind cells infected by COVID19.

[image]

[image]

[image]

### APPENDIX B

### 1. Self-Testing Kit for Private Homes

The new detection procedure is based on a kit for sample labeling and imaging using the mobile phone that will be equipped with an optical enhancement kit and special mobile application for image acquisition and analysis.

[image]

[image]

[image]

[image]

### APPENDIX B

### Self Testing Kit-Analysis

Images acquired by the mobile application will be sent to a remote analysis station.
Analyzing the image using Athena algorithm to determine the presence or absence of a fluorescence signal in the cells, wherein the presence of the signal indicates presence of the virus in the sample.

[image]

### APPENDIX B

### Self-Testing It: Mobile Application

The application will guide the user through the procedure of sample preparation
The application will control the mobile phone to acquire the images at the required imaging parameters
The application will communicate with the server to send images and facilitate analysis
The application will report the results to the user

[image]

[image]

[image]

[image]

[image]

1. A method for testing a mammalian patient for the presence of a coronavirus in the patient, the method comprising:
(1) obtaining a sample of mucus from a patient
(2) transferring the sample which has been obtained from a patient to a sample vessel, the sample vessel being substantially free of cells other than those in the sample, wherein between the obtaining of the sample and the transferring of the sample to the sample vessel the sample is not (a) incubated with other cells, (b) diluted, including diluted by contacted with a fixing solution; (c) frozen or (d) centrifuged;
(2A) fixing the cells of the sample by contacting them with a fixing solution for a time sufficient to fix the cells in the sample vessel, and thereafter conducting an initial washing of the fixed cells with a washing solution;
(2B) optionally, contacting the sample with a blocking protein, and thereafter washing the sample;
(2C) optionally, contacting the sample in the vessel with a permeabilization agent, and thereafter washing the sample;
wherein said contacting the sample with a permeabilization agent, if conducted, is conducted simultaneously with or subsequent to said contacting with a fixing solution, and wherein said contacting with a blocking protein, if conducted, is conducted simultaneously with or subsequent to said contacting with a fixing solution; and wherein, if said contacting the sample with a permeabilization agent is conducted simultaneously with said contacting with a fixing solution, said conducting an initial washing and said thereafter washing are done as a single washing step; and
wherein, if said contacting the sample with a blocking protein is conducted simultaneously with said contacting with a fixing solution, said conducting an initial washing and said thereafter washing are done as a single washing step;
(3) contacting the sample in the sample vessel with a first antibody known to bind a first viral substrate, the first viral substrate being at least one of (i) an intact first coronavirus structural protein and (ii) a portion of a first coronavirus structural protein when presented as part of a cell-surface antigen,
(4) after a first incubation period to allow binding of the first antibody to the viral substrate, conducting a first washing of the sample in the sample vessel
(5) if the first antibody is labeled with a first label, skipping to (6); if the first antibody is not labeled with a first label, after the first washing contacting the first antibody with a second antibody that binds the first antibody, the second antibody being labeled with a first label, and after a second incubation period to allow binding of the second antibody to the first antibody, conducting a second washing the sample in the sample vessel;
(6) inducing conditions which will cause the first label, if present, to produce a first signal
(7) during the time period in which the first signal would be produced if the first label is present in the sample, capturing at least one first image of the sample in the sample vessel,
(8) at least one of (a) sending the at least one first image to a remote analysis station, and (b) analyzing the at least one first image to determine the presence or absence of the first signal, wherein the presence of the first signal indicates presence of the virus in the sample.

2. The method of claim 1, the method further comprising:
(3A) contacting the sample in the sample vessel with a third antibody known to bind a second viral substrate, the second viral substrate being at least one of (i) an intact second coronavirus structural protein and (ii) a portion of a second coronavirus structural protein when presented as part of a cell-surface antigen, the second corona virus structural protein being different from the first coronavirus structural protein;
(4A) after a third incubation period to allow binding of the third antibody to the second viral substrate, conducting a third washing of the sample in the sample vessel;
(5A) if the third antibody is labeled with a second label, skipping to 6A; if the third antibody is not labeled with a second label, after the third washing contacting the third antibody with a fourth antibody that binds the third antibody, the fourth antibody being labeled with a second label, and after a fourth incubation period to allow binding of the fourth antibody to the third antibody, conducting a fourth washing of the sample in the sample vessel;
(6A) inducing conditions which will cause the second label, if present, to produce a signal
(7A) during the time period in which the second signal would be produced if the second label is present in the sample, capturing at least one second image of the sample in the sample vessel,
(8A) at least one of (a) sending the at least one second image to a remote analysis station, and (b) analyzing the at least one image to determine the presence or absence of the signal, wherein the presence of the signal indicates presence of the virus in the sample.

3. The method of claim 2, wherein steps (3) and (3A) are carried out simultaneously, the first incubation period and the third incubation period are a single incubation period, and the first and third washing are a single washing.

4. The method of claim 2 or 3, wherein both the first antibody and the third antibody are labeled with the first label and second label respectively.

5. The method of any one of claims 2 to 4, wherein the second incubation period and the fourth incubation period are a single incubation period, and the second and fourth washing are a single washing. In some embodiments, both the second antibody and the fourth antibody are labeled with the first label and second label respectively.

6. The method of any one of claims 2 to 5, wherein (7) and (7A) are carried out simultaneously, and the at least one first image and the at least one second image are the same at least one image; and (8) and (8A) are carried out simultaneously.

7. The method of any one of claim 1 to 6, wherein the method comprises contacting the sample with a blocking protein.

8. The method of any one of claim 1 to 7, wherein the first coronavirus structural protein is the spike protein.

9. The method of claim 8, wherein the second coronavirus structural protein is the nucleocapsid protein.

10. The method of claim 8, wherein the second coronavirus structural protein is the envelope protein.

11. The method of claim 8, wherein the second coronavirus structural protein is the membrane protein.

12. A method for testing a mammalian patient for the presence of a virus in the patient, the method comprising:
(b) transferring to a sample vessel a sample which has been obtained from a patient and which has not been (a) incubated with other cells (b) diluted with a solution which is not a fixing solution (c) frozen or (d) centrifuged after being obtained from said patient, the sample vessel being substantially free of cells other than those in the sample;
(c) optionally, doing one or more of the following:
(1) if the cells of the sample were not previously contacted with a fixing solution, fixing the cells of the sample by contacting them with a fixing solution for a time sufficient to fix the cells in the sample vessel, and thereafter performing an initial washing of the sample;
(2) contacting the sample with a blocking protein, and thereafter performing an initial washing of the sample;
(3) optionally, contacting the sample in the vessel with a permeabilization agent, and thereafter performing an initial washing of the sample;
wherein two or more of steps (1), (2) and (3) may be performed in parallel and, if performed in parallel, a single washing step may be employed for the steps performed in parallel;
(d) contacting the sample in the sample vessel with a first antibody known to bind a first viral substrate, the first viral substrate being at least one of an intact first viral protein, a portion of a first viral protein within a cell, and a portion of a first viral protein when presented as part of a cell-surface antigen;
(e) after an incubation period to allow binding of the first antibody to the first viral substrate, conducting a first washing of the sample in the sample vessel;
(f) if the first antibody is not labeled with a first label, after the first washing contacting the first antibody with a second antibody that binds the first antibody, the second antibody being an antibody labeled with a first label, and after an incubation period to allow binding of the second antibody to the first antibody, washing the sample in the sample vessel;
(g) inducing conditions which will cause the first label, if present, to produce a first signal;
(h) during the time period in which the signal would be produced if the first label is present in the sample, capturing at least one first image of the sample in the sample vessel; and
(i) at least one of (1) sending the first image to a remote analysis station, and (2) analyzing the first image to determine the presence or absence of the signal, wherein the presence of the signal above a predetermined threshold indicates presence of the virus in the sample.

13. The method of claim 12, further comprising:
(a) prior to said transferring, obtaining said sample from said patient.

14. The method of any one of claims 1 to claim 13, wherein the patient obtains his own sample.

15. The method of any one of claims 1 to claim 13, wherein someone other than the patient obtains the sample.

16. The method of claim 14 or claim 15, wherein said transferring is conducted by the patient.

17. The method of claim 14 or claim 15, wherein said transferring is conducted by someone other than the patient.

18. The method of any one of claims 1 to 17, wherein the sample is obtained from a nasal passage.

19. The method of any one of claims 1 to 18, wherein the sample vessel is selected from the group consisting of a 96-well plate, a petri dish and microscope slide.

20. The method of any one of claims 1 to 18, wherein the sample vessel is glass which has been coated with polylysine prior to said transferring.

21. The method of any one of claims 1 to 20, wherein said first label is a fluorescent label.

22. The method of any one of claims 12 to 21, wherein the first viral substrate is a coronavirus structural protein or a portion thereof.

23. The method of claim any one of claims 1 to 11 or claim 22, wherein the coronavirus is SARS-CoV-2.

24. The method of any one of claims 12 to 23, further comprising:
(d′) contacting the sample in the sample vessel with a third antibody known to bind a second viral substrate, the second viral substrate being at least one of an intact second viral protein, a portion of a second viral protein within a cell, and a portion of a second viral protein when presented as part of a cell-surface antigen;
(e′) after an incubation period to allow binding of the third antibody to the second viral substrate, conducting a washing of the sample in the sample vessel;
(f′) if the third antibody is not labeled with a second label, after the washing of step e′, contacting the third antibody with a fourth antibody that binds the third antibody, the fourth antibody being an antibody labeled with a second label, and after an incubation period to allow binding of the fourth antibody to the third antibody, washing the sample in the sample vessel;
(g′) inducing conditions which will cause the second label, if present, to produce a second signal;
(h′) during the time period in which the second signal would be produced if the second label is present in the sample, capturing at least one second image of the sample in the sample vessel; and
(i′) at least one of (1) sending the second image to a remote analysis station, and (2) analyzing the second image to determine the presence or absence of the second signal, wherein the presence of both the first signal and the second signal indicates presence of the virus in the sample.

25. The method of claim 24, wherein steps (i) and (i′) are carried out simultaneously.

26. The method of claim 24 or 25, wherein steps (h) and (h′) are carried out simultaneously.

27. The method of any one of claims 24 to 26, wherein the fourth antibody does not bind the first antibody or the second antibody.

28. The method of any one of claims 24 to 27, wherein said second label is a fluorescent label.

29. The method of any one of claims 24 to 28, wherein the second viral substrate is a coronavirus structural protein or a portion thereof.

30. The method of claim 29, wherein the coronavirus is SARS-CoV-2.

31. The method of any one of claims 12 to 30, wherein the sample is contacted with fixing solution prior to transfer to the sample vessel, the virus is a coronavirus, and the sample is contacted with both said first antibody which binds to the coronavirus spike protein and said third antibody which binds to the coronavirus nucleocapsid protein, membrane protein or envelope protein.

32. The method of any one of claims 12 to 30, wherein the sample is not diluted prior to transfer to the sample vessel, including diluted by contact with a fixing solution, and wherein the virus is a coronavirus.

33. The method of claim 32, wherein the sample is contacted with both said first antibody which binds to the coronavirus spike protein and said third antibody which binds to the coronavirus nucleocapsid protein, membrane protein or envelope protein.

34. The method of any one of claims 1 to 33, wherein the at least one first image is captured by a cellular telephone camera.

35. The method of any one of claims 1 to 34, wherein the method is completed in not more than 1 hour.

36. A method for reducing the likelihood of a population of persons to infect others with a coronavirus, the method comprising:
(a) obtaining a sample from each person in the population,
(a′) optionally, contacting one or more samples with a fixing solution;
(b) without (a) incubating each sample with other cells, (b) diluted each sample with a solution which is not a fixing solution, (c) freezing each sample or (d) centrifuging each sample, transferring each sample to a sample vessel, the location of each sample in the sample vessel being substantially free of cells other than those in the respective sample;
(c) optionally, doing one or more of the following:
(1) if the cells of a sample have not been previously contacted with a fixing solution, fixing the cells of the sample by contacting them with a fixing solution for a time sufficient to fix the cells in the sample vessel, and thereafter performing an initial washing of the sample;
(2) contacting a sample in the sample vessel with a blocking protein, and thereafter performing an initial washing of the sample;
(3) contacting a sample in the sample vessel with a permeabilization agent, and thereafter performing an initial washing of the sample;
wherein two or three of steps (1), (2) and (3) may be performed in series or in parallel, and if performed in parallel a single washing step may be employed;
(d) contacting each sample in the sample vessel with a first antibody known to bind a first viral substrate, the first viral substrate being one of an intact spike protein, membrane protein, envelope protein or nucleocapsid protein of a coronavirus, or a portion such protein when presented as part of a cell surface antigen or when present in an infected cell,
(e) after an incubation period to allow binding of the first antibody to the first viral substrate, conducting a first washing of each sample in the sample vessel to remove excess first antibody
(f) if the first antibody is not labeled with a first label, after the first washing contacting the first antibody with a second antibody that binds the first antibody, the second antibody being labeled with a first label, and after an incubation period to allow binding of the second antibody to the first antibody, washing each sample in the sample vessel to remove excess second antibody
(g) inducing a first set of conditions which will cause the first label, if present, to produce a first signal
(h) during the time in which the first signal would be produced if the label is present in a sample, capturing at least one first image of each sample in the sample vessel,
(i) analyzing each at least one first image to determine the presence or absence of the first signal, wherein the presence of the first signal above a threshold level indicates presence of the virus in the sample;
(j) removing those individuals determined to have the virus in their samples from the population of persons, if any such individuals are present, whereby to create a reduced population of persons, and
(k) optionally allowing the reduced population of persons to do at least one of the following: (1) disperse among the general public, (2) board a transportation vessel.

37. The method of claim 36, wherein the step of removing those individuals determined to have the virus in their samples from the population of persons comprises restricting the movement of the individuals determined to have the virus.

38. The method of claim 37, wherein the restricting the movement comprises confining the individuals to one or more designated quarantine areas.

39. The method of any one of claims 36 to 38, wherein said first antibody binds to the coronavirus spike protein or a portion therein, and the method is further characterized by:
(l) contacting each sample with a third antibody known to bind a second viral substrate, the second viral substrate being one of a membrane protein, envelope protein or nucleocapsid protein of a coronavirus, or a portion such protein when presented as part of a cell-surface antigen or a portion such protein when present in an infected cell, wherein the second viral substrate is a different viral protein or derived from a different viral protein than the first viral substrate;
the incubation period of step (d) also allowing binding of the third antibody to the second viral substrate, and the first washing also removing excess third antibody;
(m) if the third antibody is not a labeled antibody, after the first washing contacting the third antibody with a fourth antibody that binds the third antibody but not the first antibody or, if present, the second antibody, the fourth antibody being labeled with a second label, and after an incubation period to allow binding of the fourth antibody to the third antibody, washing each sample to remove excess fourth antibody;
(n) simultaneously or sequentially with inducing the first set of conditions which will cause the first label, if present, to produce a signal, inducing a second set of conditions which will cause the second label, if present, to produce a signal;
during the time in which the second signal will be produced if the second label is present in a sample, capturing at least one second image of the sample in the sample vessel, which may be the same or different from the at least one first image;
analyzing the at least one first image and the at least one second image to determine the presence or absence of the first and second signals, wherein the presence of the signals above a threshold level in the same cell in a sample indicates presence of the virus in the sample.

40. The method of any one of claims 36 to 39, wherein the method comprises allowing the reduced population of persons to board a transportation vessel, the transportation vessel being selected from the group consisting of an airplane, a helicopter, a bus, a train, a passenger or commercial ship, a boat and a ferry.

41. The method of any one of claims 36 to 41, wherein the samples are obtained from a nasal passages.

42. The method of any one of claims 36 to 41, wherein the sample vessel is selected from the group consisting of a 96-well plate, a petri dish and microscope slide.

43. The method of any one of claims 36 to 42, wherein the sample vessel is glass which has been coated with polylysine prior to said transferring.

44. The method of any one of claims 36 to 43, wherein said first label is a fluorescent label.

45. The method of any one of claims 36 to 44, wherein the virus is a coronavirus.

46. The method of claim 45, wherein the coronavirus is SARS-CoV-2.

47. The method of any one of claims 36 to 46, wherein said second label is a fluorescent label.

48. The method of any one claims 12 to 47, further comprising, prior to said capturing said at least one first image, staining at least one sample with a stain that colors cell nuclei and/or cell membranes.

49. The method of claim 48, further comprising utilizing the color resulting from the staining in the analysis of the sample.

50. The method of any one of claims 36 to 49, wherein no individuals are found to be infected with the virus and thus the reduced population is the same size as the population.

51. A kit, the kit containing:
(a) a first antibody that binds to a first viral substrate of interest;
(b) a first fluorescent or other label, which is covalently bonded to either (i) the first antibody that binds the viral substrate, or (b) a second antibody that binds to the first antibody;
(c) optionally, a third antibody that binds to a second viral substrate of interest;
(d) if the third antibody is present, a second fluorescent or other label, which is covalently bonded to either (i) the third antibody that binds the second viral substrate, or (ii) a fourth antibody which is provided and that binds to the third antibody but which does not bind the first or second antibodies; and
(e) a sample vessel.

52. The kit of claim 51, wherein each antibody is independently be provided in lyophilized form.

53. The kit of claim 50, wherein each antibody is independently provided in a vessel (1) into which a defined amount of buffer solution is added and a mixed, or (2) which contains buffer ingredients in addition to lyophilized antibody.

54. The kit of any one of claims 49 to 53, further comprising: (f) an optical apparatus for increasing the magnification of a cellphone camera or tablet computer camera; (g) a light filter that allows light at the fluorescent wavelength at which the first label fluoresces to pass through but filters out enough light at other wavelengths to facilitate acceptable detection of fluorescence by the cellphone camera or tablet computer camera; and optionally (h) a light filter that filters the excitation light.

55. The kit of any one of claims 49 to 54, further comprising a sampling apparatus, such as a cotton swab.

56. The kit of any one of claims 49 to 55, further comprising (1) one or more buffer solutions for preparing one or more antibody solutions, and for washing excess antibody, and/or (2) buffer ingredients and a mixing vessel in which the buffer ingredients and water are mixed to prepare a buffer solution.

57. The kit of any one of claims 49 to 56, further comprising (1) a solution of fixation agent or (2) dry fixation agent in a vessel to which buffer solution is added and mixed.

58. The kit of any one of claims 49 to 57, further comprising (1) a solution of permeabilization agent, or (2) dry permeabilization agent to which buffer solution is added and mixed.

59. The kit of any one of claims 49 to 58, further comprising directions for its use.

60. The kit of claim 59, wherein the directions include an internet address to which image may be transmitted for analysis, and/or from which may be downloaded one or more of (1) detailed instructions on how to use the kit and upload images, (2) software to be installed to help use the kit, (3) an analysis and report of results.

61. The method of any one of claims 36 to 50, wherein the at least one first image is captured by a cellular telephone camera.
